(19) United States
(12) Patent Application Publication
(10) Pub. No.: US 2024/0304307 A1
(43) Pub. Date: Sep. 12, 2024

(54) PSYCHOLOGICAL TEST SYSTEM AND METHOD USING OPEN PSYCHOLOGICAL SCALE EXPERT PLATFORM

(71) Applicants: Ki Nyum KIM, Uijeongbu-si, Gyeonggi-do (KR); STRENGTH COACHING PSYCHOLOGY INSTITUTE CO., LTD., Seoul (KR)

(72) Inventor: Ki Nyun KIM, Uijeongbu-si (KR)

(73) Assignees: Ki Nyun KIM, Uijeongbu-si, Gyeonggi-do (KR); STRENGTH COACHING PSYCHOLOGY INSTITUTE CO., LTD., Seoul (KR)

(21) Appl. No.: 18/271,154

(22) PCT Filed: Nov. 10, 2021
(86) PCT No.: PCT/KR2021/016294

§ 371 Date: Jul. 6, 2023

(30) Foreign Application Priority Data
Jan. 7, 2021 (KR) 10-2021-0001803

(51) Int. Cl. G16H 20/70; G10L 15/26; G16H 10/20; G16H 50/20

(52) U.S. Cl. CPC G16H 20/70; G10L 15/26; G16H 10/20; G16H 50/20

(57) ABSTRACT

A psychological test system includes: an expert platform server to which psychological scales are uploaded, and in which a user interface is provided to classify, display, and select the psychological scales, and a psychological scale DB for storing the uploaded psychological scales; a psychological test server configured to generate a testee-customized psychological test block using a psychological scale block including psychological scales selected through the expert platform server, generate and transmit customized psychological test information including an online test URL to access a customized psychological test block, to a testee terminal, and generate a test result by analyzing answers to psychological test questions through the testee terminal accessed through the online test URL; and the testee terminal configured to receive the customized psychological test information from the psychological test server, and perform answering the psychological test question of the customized psychological test block by accessing through the online test URL.

### TECHNICAL FIELD

The present disclosure relates to a psychological test system and method, and more particularly, to a psychological test system and method using an open psychological scale expert platform.

### BACKGROUND ART

A psychological test has been widely used in counseling and coaching to support psychological health of modern people. In order to develop a psychological test or a psychological scale for the same, a lot of time and cost and specialized knowledge and technology are required. Even when a psychological test is developed with such effort and cost, it is more difficult for the psychological test to enter the test market and be commercialized.

The development and commercialization of a psychological test have the follows problems. First, there are difficulties in terms of financial, technological, marketing, etc. for commercialization of the psychological test. Second, psychological test research results are not properly utilized due to the conventional exclusion policy of existing psychological test research institutes. Third, in the psychological test market, it is not easy for a newly developed psychological test to enter the market due to the exclusive possession of some psychological test distributing institutions.

Meanwhile, most psychological tests according to the related art, such as a test disclosed in Korean Patent Registration No 10-0488367, consist of an offline response type or an on-line screen test type. However, a bidirectional voice test has not been activated in the psychological tests according to the related art. Accordingly, in the psychological tests according to the related art, a testee is only a machine that responds by flickering a finger, and cannot be an active participant in the psychological test. In other words, the psychological tests according to the related art mainly adopt a simple passive response method of directly inputting text information to the questions. Thus, the existing psychological tests are unable to accurately identify the psychological state of a testee, and the existing psychological tests have a limit in providing fragmentary test results in a specific area that is predetermined in advance.

### DESCRIPTION OF EMBODIMENTS

### Technical Problem

Provided is a psychological test system and method using an open psychological scale expert platform, in which difficulties in terms of finance, technology, marketing, and the like in commercializing psychological scale research results are removed by establishing an automatic psychological test development platform for utilizing uncommercialized psychological scales, and a new concept psychological test result analysis service through a user-customized psychological scale combination is provided by providing a psychological test analysis service adopting a combination of the existing psychological scale research results.

Provided is a psychological test system and method using an open psychological scale expert platform, in which, in order to more accurately diagnose the psychological characteristics of a testee in a natural and lively way, a tester asks in voice and a testee answers in voice, and a conversation content with the testee is automatically analyzed so that various psychological characteristics of a testee, for example, strength, personality, emotion, and character of a testee, may be more actually and integrally analyzed.

### Solution to Problem

According to an embodiment of the present disclosure, a psychological test system using an open psychological scale expert platform includes: an expert platform server including an open psychological scale expert platform to which psychological scales developed by psychological scale experts are uploaded, and in which a user interface is provided to classify, display, and select the psychological scales stored corresponding to psychological testers' queries, and a psychological scale DB for storing the uploaded psychological scales; a psychological test server configured to generate a testee-customized psychological test block using a psychological scale block including psychological scales selected through the expert platform server, generate and transmit customized psychological test information including an online test URL to access a customized psychological test block, to a testee terminal, and generate a test result by analyzing answers to psychological test questions through the testee terminal accessed through the online test URL; and the testee terminal configured to receive the customized psychological test information from the psychological test server, and perform answering the psychological test question of the customized psychological test block by accessing through the online test URL including in the customized psychological test information.

In an embodiment, the psychological test server may include: a psychological scale block generation unit configured to generate a plurality of psychological scale blocks including the selected psychological scales when psychological scales corresponding to the testee's psychological test purpose are received from the expert platform server; a customized psychological test generation unit configured to provide a question selection/edition user interface for selecting and editing scale questions according to the test purpose among the plurality of psychological scale blocks, generate a testee-customized psychological test block by combining the selected question and the edited question, and transmit the customized psychological test information including an online test URL to access the customized psychological test block, to the testee terminal; a psychological test performing unit configured to, when the testee terminal accesses thereto, perform a psychological test by providing questions of the customized psychological test block corresponding to the testee terminal, to the testee terminal; and a test result generation unit configured to generate a psychological test result by analyzing answers of the psychological test performed through the testee terminal.

In an embodiment, the customized psychological test generation unit may be further configured to develop a new psychological test product by refining questions for a psychological test block including the edited and combined questions, generate a psychological test web including a test name, a customized psychological test block, a result table specification, and an online test URL to access the customized psychological test block, with respect to the developed psychological test product, and transmit the psychological test information including the online test URL to the testee terminal.

In an embodiment, the customized psychological test generation unit may include: a voice data collection unit configured to collect, as voice data, a conversation content and voice of a testee through counselling with the testee, before performing a psychological test; a psychological background information generation unit configured to generate information about testee's strength, a personality type, emotion, and character, as psychological background information, by analyzing the voice data collected by the voice data collection unit; and a test question edition unit configured to provide a user interface for re-editing and refining the psychological test questions based on the generated psychological background information.

In an embodiment, the psychological test performing unit may be further configured to, as an interface for inputting answers to a psychological test answer sheet when performing a psychological test, input answers by converting a voice file of a short sentence or a long sentence into text while answering questions through a counselor or artificial intelligence.

According to another embodiment of the present disclosure, a psychological test method using an open psychological scale expert platform includes: uploading and storing psychological scales developed by psychological scale experts through an open psychological scale expert platform provided by an expert platform server; classifying, displaying, and selecting the stored psychological scales corresponding to psychological testers' queries input through a user interface provided from the open psychological scale expert platform; when psychological scales corresponding to a testee's psychological test purpose are searched for and selected through the open psychological scale expert platform and the selected psychological scales are transmitted to a psychological test server, receiving the psychological scales and generating a psychological scale block, which are performed by the psychological test server; when scale questions according to the test purpose are selected from among the psychological scale blocks and edited through a question selection/edition user interface; generating a testee-customized psychological test block by combining the selected question and the edited question, and transmitting customized psychological test information including an online test URL to access the customized psychological test block, to a testee terminal; when the testee terminal accesses the psychological test server through the online test URL, performing a psychological test by providing questions of the customized psychological test block corresponding to the testee terminal, to the testee terminal; and generating a psychological test result by analyzing answers of the performed psychological test through the testee terminal.

### Advantageous Effects of Disclosure

According to a psychological test system and method using an open psychological scale expert platform according to the present disclosure, existing numerous psychological scales that have been developed by experts, but not utilized, may be utilized.

According to the present disclosure, psychological scale development experts may commercialize developed psychological scales, at low cost, through an online psychological test service.

According to the present disclosure, as a psychological tester establishes and utilizes useful psychological scales developed by psychological scale development experts, as a data base, a psychological test suitable for the situation or state of a testee may be customized and provided, and the testee may receive a quality psychological test service through the customized psychological test.

According to the present disclosure, in a new concept psychological test result analysis service through a user-customized psychological scale combination, as a question is asked in voice and answered in voice, a conversation content with a client is automatically analyzed, and thus, various psychological characteristics of a testee, for example, strength, a personality type, emotion, and character of a testee, may be more actually and integrally analyzed, and the psychological state and characteristics of a testee may be diagnosed and analyzed more accurately in a natural and lively way.

### BRIEF DESCRIPTION OF DRAWINGS

FIG. 1 is a block diagram of the configuration of a psychological test system using an open psychological scale expert platform, according to an embodiment of the present disclosure.

FIG. 2 is a block diagram of an example of a detailed configuration of a customized psychological test generation unit.

FIG. 3 is a block diagram of an example of a detailed configuration of an editing/refining unit.

FIG. 4 illustrates an example of a psychological test system using an open psychological scale expert platform, according to an embodiment of the present disclosure.

FIG. 5 is a flowchart showing a psychological test method using an open psychological scale expert platform, according to an embodiment of the present disclosure.

FIG. 6 illustrates an example of a psychological test method using an open psychological scale expert platform, according to an embodiment of the present disclosure.

### MODE FOR INVENTION

Hereinafter, various embodiments of the disclosure will be described with reference to the accompanying drawings. However, this is not intended to limit the disclosure to particular modes of practice, and it is to be appreciated that various modifications, equivalents, and/or alternatives that do not depart from the spirit and technical scope of the disclosure are encompassed in the disclosure.

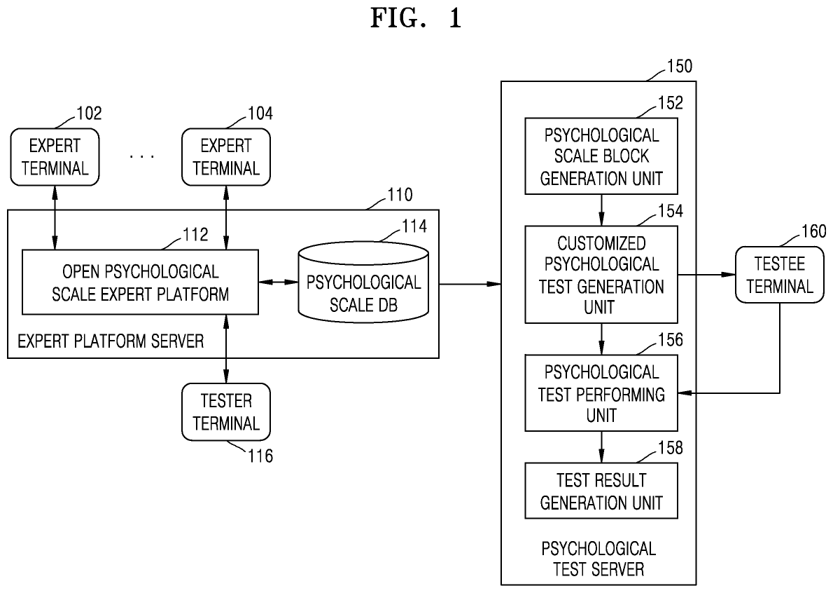

FIG. 1 is a block diagram of the configuration of a psychological test system using an open psychological scale expert platform, according to an embodiment of the present disclosure. Referring to FIG. 1, the psychological test system using an open psychological scale expert platform, according to an embodiment of the present disclosure includes an expert platform server 110, a psychological test server 150, and a testee terminal 160.

The expert platform server 110 includes an open psychological scale expert platform 112 and a psychological scale DB 114.

Psychological scales developed by psychological scale experts are uploaded to the open psychological scale expert platform 112 through expert terminals 102 and 104, and a user interface is provided to classify, display, and select the psychological scales stored corresponding to psychological testers' queries. The expert terminals 102 and 104 are connected to the open expert platform server 110 and may upload, to the open psychological scale expert platform 112, already researched and developed psychological scales, for example, a learning attitude scale, an interpersonal relationship scale, a communication scale, a learning thinking style scale, a self-esteem scale, a depression scale, a career type scale, a career maturity scale, and the like. A psychological tester may connect to the open expert platform server 110 through a tester terminal 116, and search for psychological scales provided the user interface provided through the open psychological scale expert platform 112 and select psychological scales related to the psychological test purpose among the search result.

The psychological scale DB 114 stores and manages the psychological scales developed and uploaded by the psychological scale experts. The psychological scale DB 114 may receive query of the tester terminal 116 and classify and display scales corresponding to the query.

The psychological test server 150 generates a testee-customized psychological test block by using a psychological scale block including psychological scales selected through the expert platform server 110, transmits customized psychological test information including an online test URL to access the customized psychological test block, to the testee terminal 160, and generate a test result by analyzing answers to psychological test questions through the testee terminal 160 accessed through the URL.

The psychological test server 150 may include a psychological scale block generation unit 152, a customized psychological test generation unit 154, a psychological test performing unit 156, and a test result generation unit 158.

When receiving psychological scales corresponding to the testee's psychological test purpose from the expert platform server 110, the psychological scale block generation unit 152 generates a psychological scale block including the selected psychological scales.

The customized psychological test generation unit 154 may include an editing/refining unit 210 for providing a question selection/edition user interface for selecting and editing scale questions according to the test purpose from among the psychological scale blocks, a psychological test web generation unit 220 for generating a testee-customized psychological test block by combining the selected question and the edited question, and generating and providing an online test URL to access the customized psychological test block, and a psychological test information transmission unit 230 for transmitting the customized psychological test information including the online test URL to the testee terminal 160.

The customized psychological test generation unit 154 may develop a new psychological test product by refining questions for a psychological scale block including the edited and combined questions. In this state, the editing/refining unit 210 may identify strength, a personality type, emotion, character, and the like of a testee, by analyzing conversation content and voice of the testee collected through counselling with the testee before performing a psychological test, and may re-edit and refine questions by reflecting the identified strength, a personality type, emotion, character, and the like of the testee. For example, a voice signal of a testee (client) may be classified into a frequency, a waveform, a magnitude of sound, and the like and analyzed, and the strength, a personality type, emotion, and character of the testee may be objectified based on big data. Furthermore, when counselling is performed through an face-to-face counseling on a screen, by observing facial expression changes and movements of eyes of a client, the strength, a personality type, emotion, and character of a client may be objectified based on big data. By doing so, psychological test questions may be more accurately re-edited and refined.

To this end, the editing/refining unit 210 of the customized psychological test generation unit 154 may further include a voice data collection unit 212, a psychological background information generation unit 214, and a test question edition unit 216.

The voice data collection unit 212 collects, as voice data, the conversation content and voice of a testee through counselling with the testee, before performing a psychological test. The psychological background information generation unit 214 generates information about testee's strength, a personality type, emotion, and character, as psychological background information, by analyzing the voice data collected by the voice data collection unit 212. The test question edition unit 216 provides a user interface for re-editing and refining the psychological test questions, based on the generated psychological background information.

The psychological test web generation unit 220 may generate a psychological test web including a test name, a customized scale block, a result table specification, and an online test URL to access the customized psychological test block, with respect to a developed customized psychological scale group.

The psychological test performing unit 156, when the testee terminal 160 accesses thereto, performs a psychological test by providing, to the testee terminal 160, questions of a customized psychological test block corresponding to the testee terminal 160.

The test result generation unit 158 generates a psychological test result by analyzing a psychological test answer performed through the testee terminal 160.

The testee terminal 160 receives the customized psychological test information, and accesses the psychological test server 150 through the online test URL including in the psychological test information and performs answering the psychological test question of the customized psychological test block.

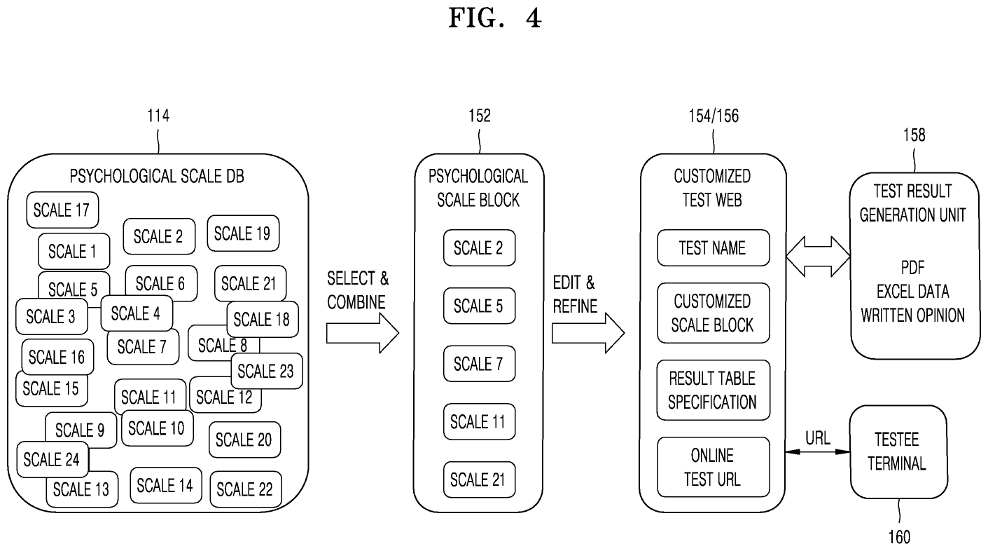

FIG. 4 illustrates an example of a psychological test system using an open psychological scale expert platform, according to an embodiment of the present disclosure. Referring to FIG. 4 the psychological scale DB 114 includes 24 psychological scales, and among them, a scale 2, a scale 5, a scale 7, a scale 11, and a scale 21 are selected and combined, and thus, the psychological scale block generation unit 152 may generate a psychological scale block. When the psychological scale block is generated, a customized psychological test is generated through the customized psychological test generation unit 154 and an online test URL for the generated customized psychological test is transmitted to the testee terminal 160. The testee terminal 160 accesses the psychological test server 150 through the URL and may perform the customized psychological test through a customized psychological test web. The test result generation unit 158 may generate a test result in a PDF file, Excel data, or a written opening and transmit the test result to the testee terminal 160.

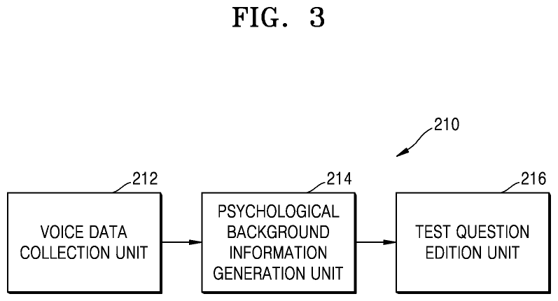

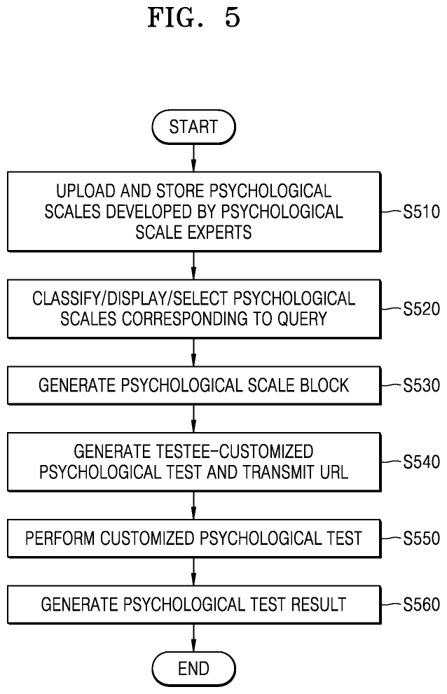

FIG. 5 is a flowchart showing a psychological test method using an open psychological scale expert platform, according to an embodiment of the present disclosure. Referring to FIGS. 1 and 3, a psychological test method using an open psychological scale expert platform, according to an embodiment of the present disclosure, is described.

Psychological scales developed by psychological scale experts are uploaded and stored through the open psychological scale expert platform 112 provided by the expert platform server 110 (operation S510).

The psychological scales stored corresponding to psychological testers' queries input through a user interface provided by the open psychological scale expert platform 112 are classified, displayed, and selected (operation S520).

Psychological scales corresponding to the testee's psychological test purpose are searched for and selected through the open psychological scale expert platform 112, and when the selected psychological scales are transmitted to the psychological test server 150, the psychological test server 150 receives the psychological scales and generates a psychological scale block (operation S530).

When scale questions according to the test purpose are selected and edited among the psychological scale blocks through the question selection/edition user interface, a testee-customized psychological test block is generated by combining the selected question and the edited question, and customized psychological test information including an online test URL to access the customized psychological test block is transmitted to the testee terminal 160 (operation S540).

When the testee terminal 160 accesses the psychological test server 150 through the online test URL, the questions of the customized psychological test block corresponding to the testee terminal 160 are provided to the testee terminal 160 and a psychological test is performed (operation S550).

A psychological test result is generated by analyzing the psychological test answer performed through the testee terminal 160 (operation S560).

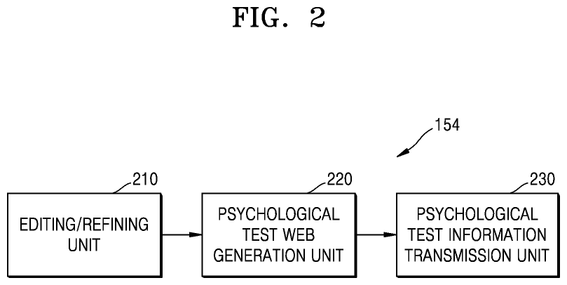

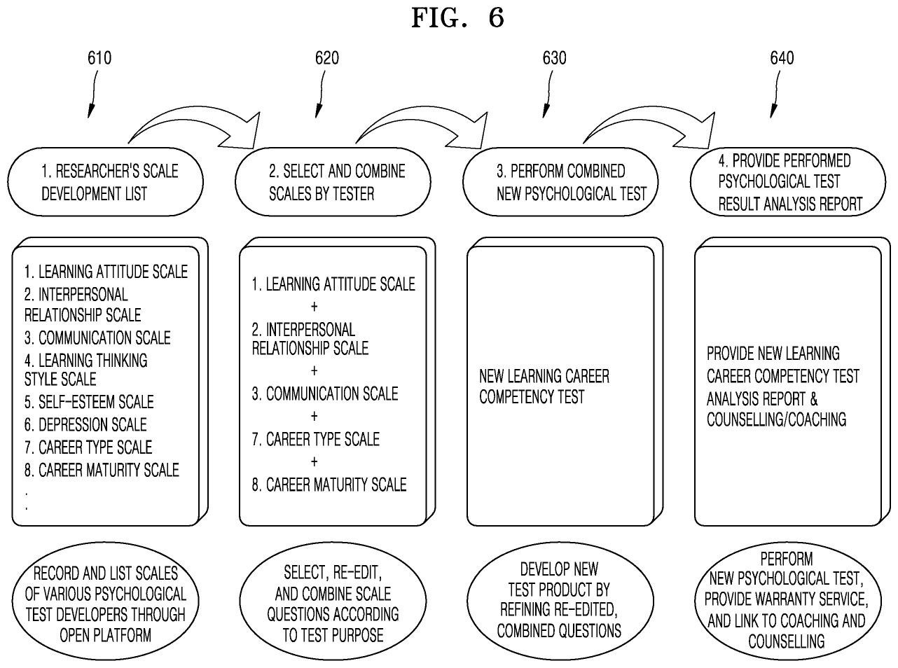

FIG. 6 illustrates an example of a psychological test method using an open psychological scale expert platform, according to an embodiment of the present disclosure. Referring to FIGS. 1 to 4, psychological scale experts or psychological scale research and developers may upload already researched and developed psychological scales, for example, a learning attitude scale, an interpersonal relationship scale, a communication scale, a learning thinking style scale, a self-esteem scale, a depression scale, a career type scale, a career maturity scale, and the like, to the open psychological scale expert platform 112, through the expert terminals 102 and 104. In other words, various psychological test scales may be recorded and listed through an open platform (610).

The psychological scale block generation unit 152 may generate a psychological scale block by selecting and combining scales according to a test purpose through the tester terminal 116 considering the situation of a testee (620). For example, a learning attitude scale, an interpersonal relationship scale, a communication scale, a career type scale, and a career maturity scale may be selected from a scale development list 610 and combined.

In this state, the customized psychological test generation unit 154 may select and re-edit scale questions according to the test purpose, and generate a customized psychological test by combining the scale questions. When the combination is completed, the psychological test performing unit 156 may organize a NEW psychological test including the combined psychological scales and perform the psychological test (630). For example, a new test product is developed by refining re-edited, combined questions, and a NEW learning career competency test may be performed.

When the psychological test is performed, the test result generation unit 158 may generate a result analysis report and provide the generated report to the testee terminal 160 (640). In other words, a NEW learning career competency test analysis report may be provided, a warranty service may be provided by using the report, and counselling and coaching may be linked.

Meanwhile, when the psychological test performing unit 156 performs a psychological test, there may be two types of methods of inputting answers to a psychological test answer sheet.

A first method is to input answers by converting voice of an answerer into a text file to output a psychological test result, and a second method is to input answers by capturing an image of an answer sheet and transmitting to output a psychological test result.

The first input method of converting voice to a text file may include inputting answers by converting the voices of YES or NO, or NUMBER ONE or NUMBER TWO with respect to psychological test questions displayed on a screen, into a text file, inputting answers by automatically converting voice into a text file while answering in a short sentence or a long sentence in the form of one-to-one conversation with an expert, or inputting answers by converting a voice file of a short sentence or a long sentence into a text while answering questions through artificial intelligence.

The second input method of capturing an image of an answer sheet may include photographing (scanning) an answered psychological test answer sheet using a smartphone and transmitting the answer sheet therethrough, and converting a transmitted image file into a text file.

While the disclosure has been particularly shown and described with reference to preferred embodiments using specific terminologies, the embodiments and terminologies should be considered in descriptive sense only and not for purposes of limitation. Therefore, it will be understood by those of ordinary skill in the art that various changes in form and details may be made therein without departing from the spirit and scope of the disclosure as defined by the following claims.

### INDUSTRIAL APPLICABILITY

The present disclosure may be used for a psychological test system and method.

1. A psychological test system using an open psychological scale expert platform, the system comprising:
an expert platform server including an open psychological scale expert platform to which psychological scales developed by psychological scale experts are uploaded, and in which a user interface is provided to classify, display, and select the psychological scales stored corresponding to psychological testers' queries, and a psychological scale DB for storing the uploaded psychological scales;
a psychological test server configured to generate a testee-customized psychological test block using a psychological scale block including psychological scales selected through the expert platform server, generate and transmit customized psychological test information including an online test URL to access a customized psychological test block, to a testee terminal, and generate a test result by analyzing answers to psychological test questions through the testee terminal accessed through the online test URL; and
the testee terminal configured to receive the customized psychological test information from the psychological test server, and perform answering the psychological test question of the customized psychological test block by accessing through the online test URL including in the customized psychological test information.

2. The psychological test system of claim 1, wherein the psychological test server comprises:
a psychological scale block generation unit configured to generate a plurality of psychological scale blocks including the selected psychological scales when psychological scales corresponding to the testee's psychological test purpose are received from the expert platform server;
a customized psychological test generation unit configured to provide a question selection/edition user interface for selecting and editing scale questions according to the test purpose among the plurality of psychological scale blocks, generate a testee-customized psychological test block by combining the selected question and the edited question, and transmit the customized psychological test information including an online test URL to access the customized psychological test block, to the testee terminal;
a psychological test performing unit configured to, when the testee terminal accesses thereto, perform a psychological test by providing questions of the customized psychological test block corresponding to the testee terminal, to the testee terminal; and
a test result generation unit configured to generate a psychological test result by analyzing answers of the psychological test performed through the testee terminal.

3. The psychological test system of claim 2, wherein the customized psychological test generation unit is further configured to:
develop a new psychological test product by refining questions for a psychological test block including the edited and combined questions; generate a psychological test web including a test name, a customized psychological test block, a result table specification, and an online test URL to access the customized psychological test block, with respect to the developed psychological test product;
and transmit the psychological test information including the online test URL to the testee terminal.

4. The psychological test system of claim 3, wherein the customized psychological test generation unit comprises:
a voice data collection unit configured to collect, as voice data, a conversation content and voice of a testee through counselling with the testee, before performing a psychological test;
a psychological background information generation unit configured to generate information about testee's strength, a personality type, emotion, and character, as psychological background information, by analyzing the voice data collected by the voice data collection unit; and
a test question edition unit configured to provide a user interface for re-editing and refining the psychological test questions based on the generated psychological background information.

5. The psychological test system of claim 2, wherein the psychological test performing unit is further configured to,
as an interface for inputting answers to a psychological test answer sheet when performing a psychological test, input answers by converting a voice file of a short sentence or a long sentence into text while answering questions through a counselor or artificial intelligence.

6. A psychological test method using an open psychological scale expert platform, the psychological test method comprising:
uploading and storing psychological scales developed by psychological scale experts through an open psychological scale expert platform provided by an expert platform server;
classifying, displaying, and selecting the stored psychological scales corresponding to psychological testers' queries input through a user interface provided from the open psychological scale expert platform;
when psychological scales corresponding to a testee's psychological test purpose are searched for and selected through the open psychological scale expert platform and the selected psychological scales are transmitted to a psychological test server, receiving the psychological scales and generating a psychological scale block, which are performed by the psychological test server;
when scale questions according to the test purpose are selected from among the psychological scale blocks and edited through a question selection/edition user interface, generating a testee-customized psychological test block by combining the selected question and the edited question, and transmitting customized psychological test information including an online test URL to access the customized psychological test block, to a testee terminal;
when the testee terminal accesses the psychological test server through the online test URL, performing a psychological test by providing questions of the customized psychological test block corresponding to the testee terminal, to the testee terminal; and
generating a psychological test result by analyzing answers of the performed psychological test through the testee terminal.
